(19) United States
(12) Patent Application Publication
(10) Pub. No.: US 2004/0265215 A1
(43) Pub. Date: Dec. 30, 2004

(54) Method and system for generating water vapor

(76) Inventors: Don DeCarli, Santa Clara, CA (US); Albert DePetrillo, Folsom, CA (US); Eric Newton, San Jose, CA (US)

Correspondence Address: Wilson & Ham, PMB: 348 2530 Berryessa Road, San Jose, CA, 95132, US

(21) Appl. No.: 10/876,115

(22) Filed: Jun. 24, 2004

Related U.S. Application Data
(60) Provisional application No. 60/483,455, filed on Jun. 26, 2003.

(51) Int. Cl. C07C 2/72; B01J 8/00

(52) U.S. Cl. 423/580.1; 422/198; 422/190; 422/211; 422/222

(57) ABSTRACT

A technique for generating water vapor involves providing oxygen and hydrogen to a heated reaction chamber that includes a porous reaction structure enclosed within an encapsulation structure. The porous reaction structure, which may include an open-celled ceramic structure, provides sufficient heat exchange and mixing to cause the oxygen and hydrogen to combine to form water vapor. The reaction chamber can be easily and safely heated using resistance, infrared lamp, radio frequency or other heating sources to a temperature above the reaction temperature required to ensure the reaction and conversion of oxygen and hydrogen to water vapor.

### CROSS-REFERENCE TO RELATED APPLICATION

This application is entitled to the benefit of provisional Patent Application Ser. No. 60/483,455, filed June 26, 2003.

### FIELD OF THE INVENTION

The invention relates to the generation of water vapor, that is used, for example, in semiconductor manufacturing operations.

### BACKGROUND OF THE INVENTION

In the manufacture of semiconductor elements, the conventional so-called dry oxygen oxidation method of silicon oxide film coating by thermal oxidations has been largely replaced by a moisture oxidation process, which is called the wet oxidation method and the steam oxidation method. The moisture oxidation method provides a silicon oxide film, which is superior to that obtained by the dry oxygen method in such properties as insulation strength and electrical characteristics.

Applicants had earlier developed a reactor for the generation of moisture by the aforesaid oxidation method. This was a supply source of high-purity water for use in silicon oxide film coating. A container of de-ionized water, a “bubbler” was utilized for the water and a very high purity carrier gas, nitrogen or oxygen, was bubbled through the de-ionized water, which created water vapor, which was then transported to the wafer processing chamber to create the oxide film.

Additional methods for creating steam have utilized pyrogenic torches to create the oxide film. Pyrogenic torches utilize very high purity oxygen and hydrogen gases at a sufficient temperature to ignite an oxygen/hydrogen flame at atmospheric pressure, producing water vapor, which is then introduced into the wafer processing chamber. This method is limited to atmospheric pressure only as a reduced atmosphere would extinguish the flame.

Another methodology involves the use of high purity oxygen and hydrogen which is injected into a reduced pressure processing chamber and utilizes the temperature of a heated wafer as the ignition source to create the steam.

Another methodology employs the utilization of a catalyst. Again oxygen and hydrogen are fed into a reactor provided with a platinum or nickel-coated catalyst layer on an interior wall, enhancing the reactivity of hydrogen and oxygen by catalytic action, and allowing the reactivity-enhanced hydrogen and oxygen to react at a temperature below the ignition point to produce moisture without undergoing combustion at a high temperature.

In the manufacturing of semiconductor devices, some conventional dry oxidation methods are being replaced by steam oxidation processes. These steam oxidation processes enable a silicon dioxide film with superior electrical and insulating properties. The current capabilities for generating wet oxide films employ pyrogenic torches, which combust oxygen and hydrogen at a sufficient temperature to ignite an oxygen/hydrogen flame at atmospheric pressure producing water vapor which is then introduced into the processing chamber. These systems are limited to atmospheric operation only since a reduced pressure condition would extinguish the flame and hence the combustion of the hydrogen and oxygen would cease.

Catalytic steam generators have been utilized for the creation of water vapor in atmospheric and reduced pressure environments. They may however, be susceptible to metallic contamination and do not have the instant on/off capability required for single wafer processing. The catalytic generators are also limited in flow rates as the hydrogen has a residence time requirement on the catalyst surface, limiting the amount of hydrogen that can be reacted. Many of the critical oxidations used in the manufacture of semiconductor devices may require the use of chlorinated gases for eliminating unwanted mobile ions within the processing chamber. The presence of chlorinated gases is incompatible with the catalyst used in the catalytic steam generators.

In-situ steam generation is also utilized to create water vapor by injecting hydrogen and oxygen into a heated processing chamber and utilizing the temperature of the wafer as the ignition source. The potential for combustion limits the processing capability to only very dilute steam concentrations within a very limited pressure range. This in turn limits the thickness of the films that can be produced. The presence of chlorinated gases is also incompatible with the stainless steel, which is typically utilized in an in-situ steam generation type of process chamber.

Requirements for ultra dilute steam (<5% concentrations) typically require downstream gas dilutions of N₂ or Ar. Since these dilutions would abort the reaction of H₂ and O₂ if passed thru the reaction chamber, additional gas plumbing, connectors and flow controllers are required, adding significant cost and complexity to the system.

### SUMMARY OF THE INVENTION

A technique for generating water vapor involves providing oxygen and hydrogen to a heated reaction chamber that includes a porous reaction structure enclosed within an encapsulation structure. The porous reaction structure, which may include an open-celled ceramic structure, provides sufficient heat exchange and mixing to cause the oxygen and hydrogen to combine to form water vapor. The reaction chamber can be easily and safely heated using resistance, infrared lamp, radio frequency or other heating sources to a temperature above the reaction temperature required to ensure the reaction and conversion of oxygen and hydrogen to water vapor.

The technique enables the production of water vapor without a combustion reaction and therefore is compatible with atmospheric as well as sub-atmospheric semiconductor manufacturing processes. Additionally, the technique enables the generation of water vapor at a wide range of concentrations and flow rates.

Other aspects and advantages of the present invention will become apparent from the following detailed description, taken in conjunction with the accompanying drawings, illustrating by way of example the principles of the invention.

### BRIEF DESCRIPTION OF THE DRAWINGS

FIG. 1 depicts a system for generating water vapor in accordance with an embodiment of the invention.

FIG. 2 depicts a system for generating water vapor that includes a single inlet system in accordance with an embodiment of the invention.

FIG. 3 depicts the system of FIG. 1 with a heat source that is in thermal communication with the reaction chamber in accordance with the invention.

FIG. 4 depicts a cross-sectional view of the reaction chamber and the heat source of FIG. 3.

FIG. 5 depicts the system for generating water vapor as depicted in FIG. 1 along with an oxygen source, a hydrogen source, and a control module.

FIG. 6 is a process flow diagram of a method for generating water vapor in accordance with an embodiment of the invention.

Throughout the description, similar reference numbers may be used to identify similar elements.

### DETAILED DESCRIPTION OF THE INVENTION

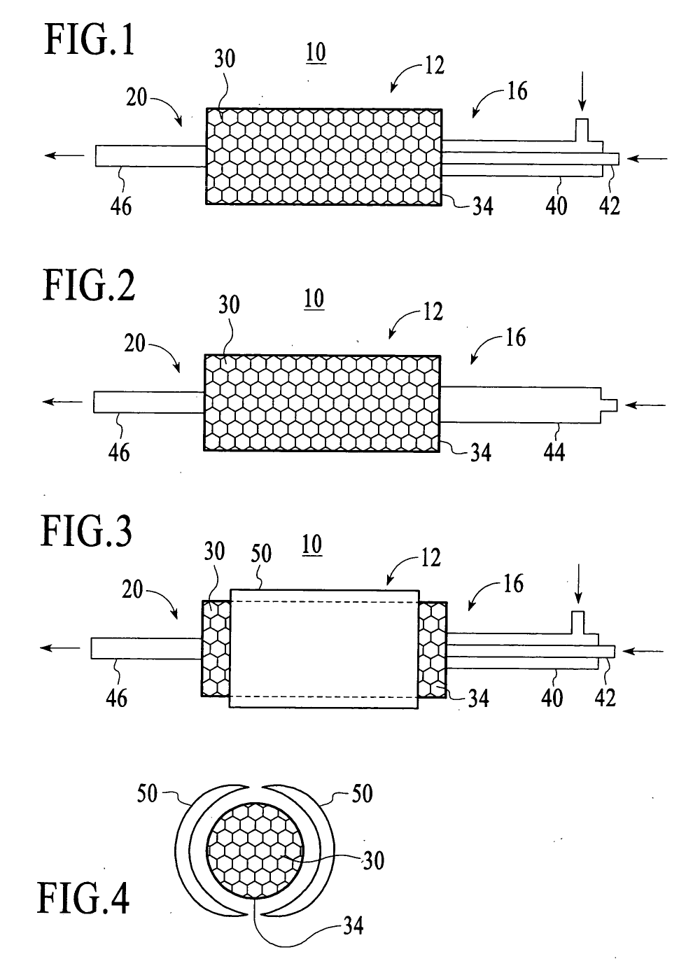

FIG. 1 depicts a system 10 for generating water vapor in accordance with an embodiment of the invention. The system includes a reaction chamber 12, an inlet system 16, an outlet system 20, and a heating source (not shown in FIG. 1). The reaction chamber includes a porous reaction structure 30 and an encapsulation structure 34. The porous reaction structure is any porous reaction structure that can provide sufficient heat exchange and gas mixing to support the reaction of oxygen and hydrogen to water vapor. In an embodiment, the porous reaction structure is a ceramic material that is formed into an open-celled structure. The porous reaction structure can also be described as a three-dimensional latticework of interconnected ceramic ligaments. In an embodiment, the porous material is made by ERG Materials and Aerospace Corporation, Oakland, Calif. and sold under the trademark DUOCEL. In an embodiment, a DUOCEL silicon-carbide (SiC) product with 8% nominal density is used. This DUOCEL SiC product has the characteristics shown in Table 1.

TABLE 1
DUOCEL Characteristics

| Characteristic | English Units | Metric Units |
|---|---|---|
| Compression Strength | 200 psi | (1.38 MPa) |
| Flexural Strength | 400 psi | (2.76 MPa) |
| Shear Strength | 100 psi | (.69 MPa) |
| Young's Modulus | 4 · 10⁵ psi | (2.76 GPa) |
| Knoop Hardness (100 gm) | 2500 | |
| Poisson's Ratio | 0.22 | |
| Thermal Conductivity: | | |
| 482° F. (250° C.) | 3.05 BTU · ft⁻¹ · hr⁻¹ · ° F.⁻¹ | (5.28 W · m⁻¹ · ° C.⁻¹) |
| 1832° F. (1000° C.) | 1.07 BTU · ft⁻¹ · hr⁻¹ · ° F.⁻¹ | (1.85 W · m⁻¹ · ° C.⁻¹) |
| 2642° F. (1450° C.) | .77 BTU · ft⁻¹ · hr⁻¹ · ° F.⁻¹ | (1.34 W · m⁻¹ · ° C.⁻¹) |
| Thermal Expansion Coefficients: | | |
| Room Temperature | 1.22 · 10⁻⁶ in · in⁻¹ · ° F.⁻¹ | (2.2 · 10⁻⁶ m · m⁻¹ · ° C.⁻¹) |
| 392° F. (200° C.) | 2.06 · 10⁻⁶ in · in⁻¹ · ° F.⁻¹ | (3.7 · 10⁻⁶ m · m⁻¹ · ° C.⁻¹) |
| 932° F. (500° C.) | 2.56 · 10⁻⁶ in · in⁻¹ · ° F.⁻¹ | (4.6 · 10⁻⁶ m · m⁻¹ · ° C.⁻¹) |
| 1292° C. (700° C.) | 2.72 · 10⁻⁶ in · in⁻¹ · ° F.⁻¹ | (4.9 · 10⁻⁶ m · m⁻¹ · ° C.⁻¹) |
| Bulk Resisitivity at Room Temperature | 4 · 10⁵ ohm · in | (1.6 · 10⁵ ohm · cm) |
| Sublimation Point | 4892° F. | (2700° C.) |
| Max Continuous Use (Inert | 3992° F. | (2200° C.) |
| Atmosphere) | | |
| Oxidation Resistance | 3002° F. | (1650° C.) |

The porous reaction structure 30 is configured such that gases can flow through the material with a resistance to flow that is proportional to the nominal density of the ceramic material. Although a SiC ceramic material is described as the porous reaction structure, other materials that exhibit properties similar to those in Table 1 could be used for the porous reaction structure.

The encapsulation structure 34 encapsulates the porous reaction structure 30 to control and direct the flow of gas through the porous reaction structure. In an embodiment, the encapsulation structure is quartz (e.g., General Electric 214 quartz or an equivalent). Although quartz is described as the encapsulation material, other materials, for example, SiC, alumina, sapphire, or other ceramic-like materials can be used to form the encapsulation structure. The encapsulation structure also includes openings that form the inlet and outlet systems or some portion thereof.

In the embodiment of FIG. 1, the inlet and outlet systems 16 and 20 are connected to the encapsulation structure 34. The inlet system includes quartz tubes that are fused to the encapsulation structure. In the example of FIG. 1, the inlet system includes separate tubes 40 and 42 for the oxygen and hydrogen. For example, the hydrogen tube 42 runs within the oxygen tube 40. The separate tubes keep the oxygen and hydrogen gases separate until they enter the reaction chamber 12. Although the hydrogen tube is within the oxygen tube in the example of FIG. 1, the tubes could be parallel to each other and attached to different locations on the encapsulation structure. Alternatively, as shown in FIG. 2, the oxygen and hydrogen could be mixed in a single inlet tube 44 before being provided to the reaction chamber. The inlet system may include a single gas delivery tube or multiple gas delivery tubes depending on the configuration.

The outlet system 20 is any structure that allows the generated water vapor to exit the reaction chamber 12. For example, the outlet system may include a quartz tube 46 fused to the encapsulation structure as depicted in FIG. 1. Although the inlet and outlet systems are described herein as including quartz tubes, the inlet and outlet systems could consist simply of openings in the encapsulation structure 34 through which the gases would enter and exit the reaction chamber. Additional gas flow systems would then be attached to the openings in the encapsulation structure.

The heat source of the system provides heat to the reaction chamber. For example, the heat source should be able to heat the reaction chamber to 580 degrees Celsius and above to support the generation of water vapor from the combination of oxygen and hydrogen gases. The heat source can be any mechanism that provides heat to the reaction chamber. Example heat sources include resistive, infrared (IR), and radio frequency (RF) heat sources. FIG. 3 depicts the system of FIG. 1 with a heat source 50 that is in thermal communication with the reaction chamber. In the example of FIG. 3, the heat source is a clamshell-type resistive heater although the specific type of heat source is not critical.

FIG. 4 depicts a cross-sectional view of the reaction chamber and the heat source 50 of FIG. 3. The cross-sectional view depicts the porous reaction structure 30 and the encapsulation structure 34. Although the system is shown in FIG. 3 as having a circular cross-section, other cross-sectional shapes can be used.

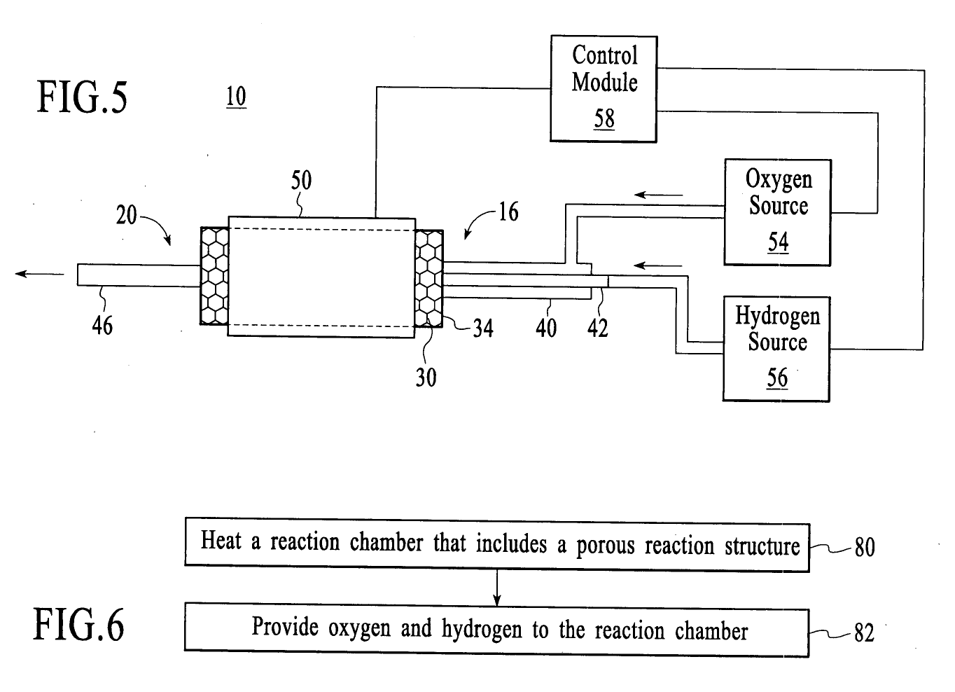

FIG. 5 depicts the system 10 for generating water vapor as depicted in FIG. 1 along with an oxygen source 54, a hydrogen source 56, and a control module 58. In an embodiment, the oxygen and hydrogen sources provide oxygen and hydrogen at the desired process conditions in terms of, for example, temperature, pressure, and concentration. The control module controls the temperature of the reaction chamber and the flow rate of the oxygen and the hydrogen. The control module can be implemented with any combination of hardware, software, or firmware as is known in the field.

In operation, the heat source 50 is controlled to heat the reaction chamber 52 to the desired temperature. Once the reaction chamber is heated to the desired temperature (e.g., greater than 580 degrees Celsius), oxygen is allowed to flow from the oxygen source 54 to the reaction chamber. After the oxygen is flowing to the reaction chamber, the hydrogen is allowed to flow from the hydrogen source 56. At the reaction chamber, the oxygen and hydrogen are mixed and heated until they combine to form water vapor. The resulting water vapor then exits the reaction chamber through the outlet system 20 where it can be delivered for its intended use, for example, in semiconductor manufacturing processes.

FIG. 6 is a process flow diagram of a method for generating water vapor in accordance with an embodiment of the invention. At step 80, a reaction chamber that includes a porous reaction structure is heated. At step 82, oxygen and hydrogen are provided to the reaction chamber.

A system and method of generating water vapor of any concentration for use in semiconductor processing, consisting of feeding oxygen and hydrogen gases through a porous ceramic material such as silicon carbide, aluminum oxide, sapphire and or other like materials surrounded by a quartz or ceramic wall and connected to a tube of like material to allow the flow of gases through the porous material. The porous ceramic material is heated using resistance, IR lamp, RF or other heating sources to a temperature above the reaction temperature required to insure the reaction and conversion of hydrogen and oxygen to water vapor, which is then delivered to a thermal chamber or tube to enable the silicon oxide film to be grown. Individual gas injectors for hydrogen and oxygen are designed to ensure safe and complete conversion to water vapor in atmospheric or sub-atmospheric conditions. The generation of water vapor is typically started by feeding oxygen into the apparatus followed by the introduction of hydrogen at the site of the heated ceramic material thus ensuring the safe conversion to water vapor within the heated ceramic material. The water vapor generation process is typically terminated by first shutting off the supply of hydrogen followed by shutting off the oxygen supply. This process enables the production of water vapor in atmospheric and sub-atmospheric conditions without necessitating the combustion reaction heretofore used. The apparatus attaches to atmospheric or sub-atmospheric processing chambers or batch systems to allow wet or steam oxidations for semiconductor manufacturing. The process may be referred to as water vapor oxidation method, a moisture oxidation method, a wet oxidation method, or a steam oxidation method.

This technique provides a safe method for generating steam at atmospheric or reduced pressure for the manufacture of semiconductor devices. This technique has no flow limitations, has infinite steam concentration capabilities from less than 1 to 100%. It also operates in any reduced pressure range from less than 100 millitorr to atmospheric pressure. The ceramic “foam” enables the desired hydrogen/oxygen reaction for reduced pressure steam generation without dangerous combustion reactions and is completely compatible with chlorinated gases. Unlike other water vapor generators, downstream dilutions of N₂ or Ar for ultra dilute steam requirements (<5% concentrations) are not necessary since all gases can be passed thru the reaction chamber to achieve the required dilution with no reduction in steam generation capability.

An example system includes two distinct parts, 1) being the Silicon Carbide “foam” or the heating element, and 2) the encapsulation to hold the “foam” and provide a gas passageway. The DUOCEL Silicon Carbide “foam” is a porous, open-celled structure. It is made of a three dimensional latticework of interconnected ceramic ligaments. The DUOCEL ceramic material is solid, fine grained, Beta phase (cubic crystal) Silicon Carbide. The “foam” itself is composed of reticulated vitreous carbon. It is a form of glass-like carbon, which combines some of the properties of glass with some of those of normal industrial carbons. The “foam” can be fabricated in various pore sizes (10-100 pores per linear inch “ppi”) and various relative silicon carbide coating densities from (e.g., between 3%-30%). The “foam” is machined to the required shape and dimensions. The machined “foam” is then placed in a chemical vapor deposition chamber for the coating process, where a coating of silicon carbide is deposited on the surface. Additional coatings of silicon/silicon nitride or other like materials may also be deposited. For atmospheric processing, “foam” diameters of, for example, ≧2.0 inches are utilized to allow for the exothermic reaction to be contained. For sub-atmospheric processing, the foam diameter can be reduced to, for example, ≦1.0 inches as the exothermic reaction at reduced pressure is substantially less.

Once the “foam has been machined and coated, it is ready for encapsulation. The material used for encapsulation is quartz (GE 214 or equivalent), but other materials may be used, i.e. silicon carbide, alumina, sapphire or other like ceramics. The “foam” is encapsulated into the reaction chamber and then the outlet and inlet tubes are attached. This is typically done at a quartz or glassblowing supplier. The outlet and inlet tubes are made of the same material as the reaction chamber. An additional injector is utilized (identified as the hydrogen inlet in FIG. 1.) for safety purposes. This injector is placed in the middle of the gas inlet tube and is made of quartz, alumina, sapphire, silicon carbide or other like ceramic material. This injector serves to keep the two gases—hydrogen and oxygen—separated until such time as they are introduced to the activation energy in the foam, to create the water vapor.

The encapsulated foam itself is heated via a resistance heater, IR lamp, or RF generator. In this instance, a clamshell resistance heater is utilized to create the activation energy, although other types are available. The activation energy for hydrogen is approximately 580° C. In one example, the heaters are set at ≧700° C. to ensure that the activation energy is sufficient to enable the reaction of the H₂ and O₂ to form steam. Upon reaching this energy level, a flow of oxygen is released into the inlet tube. After oxygen has started flowing, hydrogen is then introduced into the reaction chamber, whereupon it combines with the oxygen above the activation energy level and water vapor is created. This water vapor is then carried to the process chamber where it reacts with silicon substrates to create the desired silicon dioxide film.

At atmospheric pressure, the typical temperature set point is ≧700° C. In one example, the flow rates can range from approximately 100 standard cubic centimeters per minute (SCCM) to approximately 30 standard liters per minute (SLPM) depending on water vapor concentrations and flow requirements. A nitrogen or argon diluent gas can also be passed thru the “foam” and is typically used when ultra-dilute water vapor is required for very thin oxidation films. The nitrogen or argon can be passed through either the oxygen or hydrogen inlet or a separate inlet and can be mixed with either of the gases with no effect on the process other than to dilute the mixture. Chlorinated gases may also be passed thru either inlet or a separate inlet for the purpose of eliminating unwanted mobile ions in the process chambers.

At sub-atmospheric pressures, the temperature ranges remain the same as atmospheric (≧700° C.). In one example, the flows are reduced from the atmospheric operation flows in order to accommodate the low-pressure process requirements. Flows may range, for example, from approximately 10 SCCM to 1,000 SCCM of either gas in any combination and pressure ranges between approximately 100 millitorr and atmospheric pressure, depending on the application.

Although specific embodiments of the invention have been described and illustrated, the invention is not to be limited to the specific forms or arrangements of parts as described and illustrated herein. The invention is limited only by the claims.

What is claimed is:

1. A system for generating water vapor comprising:
a reaction chamber comprising:
a porous reaction structure; and
an encapsulation structure that encapsulates the porous reaction structure,
the encapsulation structure including an inlet system and an outlet system;
a heat source in thermal communication with the reaction chamber configured to heat the reaction chamber.

2. The system of claim 1 wherein the porous reaction structure is formed of ceramic.

3. The system of claim 1 wherein the porous reaction structure is an open-celled ceramic structure.

4. The system of claim 1 wherein the porous reaction structure is DUOCEL.

5. The system of claim 1 wherein the porous reaction structure is formed of a silicon carbide open-celled structure.

6. The system of claim 1 wherein the porous reaction structure is formed of a pattern of ceramic cells and ligaments.

7. The system of claim 1 wherein the porous reaction structure is formed of silicon carbide.

8. The system of claim 1 wherein the inlet system includes separate inlets for receiving oxygen and hydrogen.

9. The system of claim 1 wherein the inlet system includes a common inlet for oxygen and hydrogen.

10. The system of claim 1 wherein the encapsulation structure is formed of quartz.

11. The system of claim 10 wherein the inlet and outlet systems include quartz tubes that are fused to the encapsulation structure.

12. A system for generating water vapor comprising:
a reaction chamber comprising:
a porous ceramic reaction structure; and
an encapsulation structure that encapsulates the porous ceramic reaction structure, the encapsulation structure including an inlet system configured to receive hydrogen and oxygen into the reaction chamber and an outlet system configured to output water vapor; and
a heat source in thermal communication with the reaction chamber configured to heat the reaction chamber;
wherein oxygen and hydrogen can be heated and mixed within the reaction chamber to generate water vapor.

13. The system of claim 12 wherein the porous reaction structure is an open-celled ceramic structure.

14. The system of claim 12 wherein the porous reaction structure is DUOCEL.

15. The system of claim 12 wherein the porous reaction structure is formed of a silicon carbide open-celled structure.

16. A method for generating water vapor comprising:
heating a reaction chamber that includes a porous reaction structure; and
providing oxygen and hydrogen to the reaction chamber.

17. The method of claim 16 wherein the porous reaction structure is an open-celled ceramic structure.

18. The method of claim 16 wherein the porous reaction structure is DUOCEL.

19. The method of claim 16 wherein the porous reaction structure is formed of a silicon carbide open-celled structure.

20. The method of claim 16 wherein the oxygen and hydrogen are provided to the reaction chamber separately.

21. The method of claim 16 wherein the oxygen and hydrogen are provided to the reaction chamber as a mixture.

22. The method of claim 16 wherein the reaction chamber is heated to a temperature of 580 degrees Celsius.

23. The method of claim 16 wherein, at atmospheric pressure:
the oxygen is provided at between 100 standard cubic centimeters per minute and 20 standard liters per minute; and
the hydrogen is provided at between 100 standard cubic centimeters per minute and 20 standard liters per minute.

24. The method of claim 16 wherein, at sub-atmospheric pressure:
the oxygen is provided at between 10 standard cubic centimeters per minute and 1 standard liters per minute; and
the hydrogen is provided at between 10 standard cubic centimeters per minute and 1 standard liters per minute.

25. The method of claim 16 wherein the oxygen is provided to the reaction chamber before the hydrogen.
